(19) United States
(12) Patent Application Publication
(10) Pub. No.: US 2023/0236206 A1
(43) Pub. Date: Jul. 27, 2023

(54) PREDICTING A SEPSIS CONDITION

(71) Applicant: PHARM-ANALYT LABOR GMBH, BADEN (AT)

(72) Inventor: HERMANN MASCHER, TRAISKIRCHEN (AT)

(21) Appl. No.: 17/999,729

(22) PCT Filed: May 5, 2021
(86) PCT No.: PCT/EP2021/061797

§ 371 Date: Nov. 23, 2022

(30) Foreign Application Priority Data
May 25, 2020 (EP) 20176330.7

(51) Int. Cl. G01N 33/68

(52) U.S. Cl. CPC G01N 33/6893; G01N 2800/26; G01N 2800/50; G01N 2800/52

(57) ABSTRACT

A method of predicting a sepsis condition in a subject, comprising: a. determining a level of a biomarker in a sample of said subject, wherein the biomarker is of structure (I): Formula (I) or a salt thereof; and b. comparing said level to a predetermined reference value of the biomarker, wherein an elevated biomarker level is indicative of the risk of the sepsis condition.

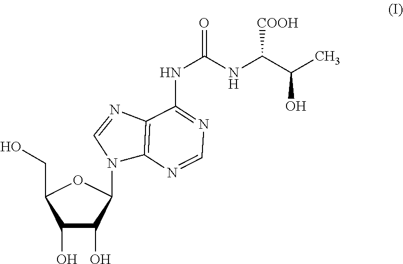

The invention refers to a method of predicting a sepsis condition in a subject, which comprises determination of a biomarker.

### BACKGROUND

Sepsis is defined as a life-threatening organ dysfunction caused by a dysregulated response of the host to an infection. According to the most recent estimates, sepsis cases approximated 49 million and sepsis-related deaths approximated 11 million worldwide. Current definition of sepsis recognizes two stages of sepsis: sepsis and septic shock. Predicting sepsis and its progression is a challenge, and the diagnosis is frequently made too late for respective treatment to prevent the life-threatening organ dysfunction and subsequent death. An early and accurate identification of a patient's potential for sepsis development in a high-risk population e.g. a hospitalized patient, in particular an ICU patient, is of significant clinical value. Conversely, ruling out sepsis development is of equal importance in terms of withholding antibiotics (to avoid development of secondary antibiotic-resistant infections), and conducting additional sepsis-related investigations.

Current diagnostic methods aim to differentiate between the systemic inflammatory response syndrome (SIRS) and sepsis. Procalcitonin (PCT) is a common biomarker used to diagnose bacterial infections and distinguish them from non-infectious SIRS-like conditions. However, an accurate measure of disease severity has not yet been developed and depends solely on clinical scores such as APACHE, SAPS and SOFA. Cut-off levels of PCT indicating the likelihood of a bacterial infection are debated and ambiguous regarding their application in the clinic. According to current practice, clinical ICUs use varying PCT cut-offs; either 0.25 ng/mL, 0.5 ng/mL or 1.0 ng/mL in plasma as a threshold to start antibiotic intervention and rule in/out an infection. Varying cut-offs constitute a trade-off between sensitivity and specificity; for example, the use of PCT at its lower cut-offs to diagnose sepsis carries a substantial risk of false positives (no bacterial infection despite PCT elevation), especially in cases of multiple trauma, severe burns, major surgeries, malaria patients and in newborns (at least up to the fourth day after birth).

WO84/04168A1 discloses methods and a test kit for diagnosing cancer in humans. An exemplary test kit comprises monoclonal antibodies specifically recognizing N-[9-(β-D-ribofuranosyl)purin-6-ylcarbamoyl]-L-threonine.

Ludwig et al. (Molecular Biosystems 2017, 13(4): 648-664) describe mass spectrometry methods for the discovery of biomarkers of sepsis.

WO2004/044554A2 discloses the diagnosis of sepsis or SIRS using biomarker profiles.

It can be critical to provide a reliable diagnostic test for early diagnosis of sepsis (in particular in patients with high risk of developing sepsis) and for the purpose of antibiotic therapy guidance (both initiation and discontinuation), stratification and/or monitoring of patients suffering from an infectious disease and receiving treatment with one or more antimicrobial agents.

### SUMMARY OF THE INVENTION

It is an objective to provide means and methods for determining sepsis and/or the onset of sepsis disease, in particular in a patient of a risk population, such as those suffering from an infectious disease and presenting with pre-existing comorbidities.

The objective is solved by the subject matter of the present claims and as further described herein.

The invention provides for a method of predicting a sepsis condition in a subject, comprising:
a. determining a level of a biomarker in a sample of said subject, wherein the biomarker is of structure (I) or a salt thereof, as further described herein; and
b. comparing said level to a predetermined reference value of the biomarker, wherein an elevated biomarker level is indicative of the risk of the sepsis condition.

Specifically, the biomarker is a small molecule compound that is endogenous or naturally-occurring in the sample. The biomarker of structure (I) or a salt thereof, can be any respective (one or more e.g., the total of) optical isomer, enantiomer, diastereomer, or racemate, to be determined in the sample.

Specifically, the reference level is the level of the biomarker in a subject or a group of subjects not being at said risk, or a threshold level indicating the sepsis condition.

The reference level may be a value resulting from calibrating the method against samples of patients with a known disease or risk of disease.

Specifically, the methods described herein may be quantitative or semi-quantitative methods such as determining whether the biomarker level in the sample is above a reference or threshold level.

A threshold level (also referred to as cut-off level) may distinguish between healthy subjects and those subjects who are at a higher risk of developing disease, at the onset of disease, or already suffering from disease.

Specifically, the method described herein is used for therapy guidance, stratification and/or control, in particular antibiotic therapy. To this end, the method described herein specifically further comprises applying, maintaining, reducing, elevating or not applying a therapy based on whether the subject is at said risk.

Specifically, the sepsis condition is any one or more of sepsis diseases selected from the group consisting of systemic inflammatory response syndrome (SIRS), sepsis, septic shock and multiple organ dysfunction syndrome (MODS).

According to a specific aspect, the method described herein is used to determine the biomarker in a subject not suffering from said sepsis condition, and the onset of such sepsis condition is predicted based on the level of biomarker in the sample.

Specific sepsis conditions which are understood as being sepsis include e.g. any one or more of sepsis diseases selected from the group consisting of sepsis (from different sources and of different origin), septic shock and multiple organ dysfunction syndrome (MODS; as an underlying component in the new Sepsis-3 definition, Singer et al. 2016).

Specifically, the subject is an infected patient, i.e. a patient diagnosed with infection or suffering from probable or confirmed infection.

In certain embodiments, the risk of a sepsis condition is indicated in an infected patient if the biomarker level is higher than a predetermined reference level normally found in the patient population that does not suffer from such infection, or that does not develop such sepsis condition.

The predetermined reference level in a certain assay that is higher than a normal level can be e.g., a level which is at least 2-fold, 3-fold, 4-fold, or 5-fold higher than the standard deviation of measuring the normal level.

For example, the predetermined reference level in blood, plasma or serum samples can be a threshold of about 15-40 ng BM1/mL, such as about 15, 20, 25, 30, 35, or 40 ng BM1/mL.

Values significantly higher than such threshold (or at least higher than the standard deviation) would indicate the risk of a sepsis condition. The present examples employing a certain BM1 test and comparing human patients suffering from sepsis versus a control group suffering from heart failure establish a threshold of about 32 ng BM1/mL of plasma or serum (example 4, matrix: sensitivity/specificity).

The predetermined reference level in plasma or serum samples of human newborns can be about 80-120 ng BM1/mL. Septic human newborns may have an elevated level of up to 300 ng/mL in plasma or serum.

The predetermined reference level in blood, plasma or serum samples of calves, lama, alpaca, goat, or beef is about 10-30 ng/mL, some septic calves showed concentration levels of about 40-80 ng/mL for BM1.

The predetermined reference level in synovial fluid of human beings can be about 15-40 ng BM1/mL, some septic human beings showed concentration levels of about 40-160 ng/mL for BM1.

The predetermined reference level in human urine can be a threshold of about 2500-4500 ng BM1/mg Creatinine.

Septic human patients typically have an elevated level of about 5000-16000 ng BM1/mg Creatinine.

A case of septic shock is specifically indicated if the biomarker level is at least any one of 10%, 20%, 30%, 40%, 50%, 60%, 70%, 80%, 90%, or 100% higher than the reference level indicating sepsis.

The invention further provides for a method of predicting a sepsis condition in a subject not suffering from said sepsis condition, comprising:
a. determining a level of a small molecule biomarker in a sample of said subject, and
b. comparing said level to a predetermined reference value of the biomarker, wherein an elevated biomarker level is indicative of the risk of the sepsis condition onset.

The invention further provides for a method of monitoring a sepsis condition in a subject not suffering from said sepsis condition, comprising:
a. determining the level of a small molecule biomarker in a sample of a subject at a first time point, and
b. determining the level of the biomarker in a sample of the same subject at a later second time point,

wherein an increase in the level of the biomarker between the first and second time points is indicative of a sepsis condition onset.

Specifically, the risk of a sepsis condition onset is indicated if the biomarker level is higher than expected for the subject. In particular, if the subject is an infected patient, the onset of a sepsis condition is indicated or predicted, if the biomarker level in the sample is higher than a predetermined reference level normally found in the patient population that does not suffer from a bacterial infection, or that does not develop such sepsis condition. Such predetermined reference levels can be used as further described herein.

Usually, the higher the biomarker level in the test sample, the higher the likelihood for a fast onset of the disease, or disease progression.

Specifically, the biomarker is a small molecule biomarker endogenous to the sample originating from the subject, preferably of structure (I) or a salt thereof, as further described herein.

Specifically, the biomarker described herein is of structure (I) or a salt thereof, and may be any one or more (or all) of the isomers (stereoisomers) of structure (I) or a salt thereof, or a mixture of one or more of such isomers, e.g., an optical isomer, enantiomer, diastereomer, or racemate, each characterized by the structure (I) or a salt thereof. According to a particular example, all structural conformations of the biomarker are determined and the respective biomarker level represents the total concentration of compounds of structure (I) or a salt thereof.

Specifically, the biomarker is determined applying an analytical method selected from the group consisting of an immunoassay, chromatography, preferably HPLC chromatography, UPLC chromatography, gas chromatography (GC) or thin layer chromatography, capillary electrophoresis, mass spectrometric analysis or NMR spectroscopy.

Specifically, the analytical method employs UV, Fluorescence, MS or MS/MS detection, or detection of a label, preferably an enzymatic label, a fluorescent label, or a radioisotope label.

Specifically, mass spectrometric analysis is selected from the group consisting of SELDI, MALDI, MALDI-Q TOF, MS/MS, TOF-TOF and ESI-Q-TOF.

Specifically, the immunoassay is selected from the group consisting of an enzymatic immunoassay, such as an ELISA (e.g. a sandwich ELISA), lateral flow immunochromatographic assay, fluorescent immunoassay, radioimmunoassay, and magnetic immunoassay.

Specifically, the sample is a biological sample, such as a body fluid selected from the group consisting of blood, plasma, serum, urine, faeces, sputum, synovial fluid and saliva.

Specifically, the sample is prepared before testing by one or more suitable techniques to purify the sample or to separate any interfering matter, such as separation of cells, removal of proteins by e.g., protein precipitation, or by dilution.

Urine samples may be diluted before testing e.g., 1:20 or 1:50 diluted.

Serum or plasma samples may be treated to precipitate proteins before testing.

Blood samples may be treated to precipitate proteins before testing.

For HPLC-MS/MS determination the serum (or plasma or blood) proteins may be removed by precipitation using e.g., acetonitrile. After centrifugation and hence separation of precipitate from clear supernatant, part of the supernatant can be used for HPLC-MS/MS determination.

A sample can be collected at different time points e.g., at hospital administration, or at the onset of clinical symptom(s) of an infection, and freshly used or stored before analyzing e.g., at room temperature, or upon freezing.

Specifically, the sample is isolated from said subject within 48 hours, preferably within 24 hours, more preferably within 6 hours after showing at least one or two early symptoms of sepsis, at least one or two qSOFA points found in an infected patient.

Specifically, the subject is a human being or a non-human animal subject, in particular a vertebrate, preferably a dog, horse, camel, cow, cat, rabbit, pig or rat.

According to a specific embodiment, the subject is a human patient who is of a patient group with a high risk of developing sepsis or septic shock, in particular a hospitalized patient. Specifically, the human patient is an adult, child, infant or newborn.

According to a specific embodiment, the subject is a non-human animal e.g., an animal used for experimental methods of disease treatment, such as used in animal models.

The present biomarker may be used for monitoring or surveillance. For example, the biomarker can be used for surveillance purposes supporting the physician predict or monitor the progression of disease.

The invention further provides for a method of monitoring a sepsis disease in a subject, comprising:
a. determining the level of a biomarker in a sample of a subject at a first time point, wherein the biomarker is of structure (I) or a salt thereof, as further described herein; and
b. determining the level of the biomarker in a sample of the same subject at a later second time point,

wherein an increase in the level of the biomarker between the first and second time points is indicative of the sepsis disease progression, or indicative of a higher risk of any subsequent sepsis development.

Specifically, in such method, the sample is of an infectious disease patient that has been identified as a low risk patient by conventional means, and an increase in the level of the biomarker indicates the risk of a more severe sepsis condition, or developing a sepsis disease, or be indicative of a subsequent sepsis event, e.g. expected within a short timeframe e.g. within hours or within 1, 2, 3 or 4 days after the first or second sampling, in particular, unless the patient is appropriately treated to prevent such disease progression.

The invention further provides for a method of monitoring the effectiveness of at least one treatment applied to a subject for a sepsis condition, comprising:
a. determining the level of a biomarker in a sample of a subject at a first time point, wherein the biomarker is of structure (I) or a salt thereof, as further described herein; and
b. determining the level of the biomarker in a sample of the same subject at a later second time point, and

wherein a decrease in the level of the biomarker between the first and second time points is indicative of treatment success, or indicative of a lower risk of any subsequent sepsis development.

Specifically, in such method, the sample is of an infectious disease patient that has been identified as a high risk patient or sepsis patient by conventional means, and a decrease in the level of the biomarker indicates the risk of a less severe sepsis condition, or ameliorating a sepsis disease or condition, or be indicative of a subsequent cure of a sepsis disease or condition, e.g. expected within a short timeframe e.g. within hours or within 1, 2, 3 or 4 days after the first or second sampling, in particular, if treatment continues.

Specifically, the treatment is medical treatment such as antibiotic treatment e.g. by intravenous antibiotics, oral antibiotics or topical antibiotics.

Specifically, the first and second samples are of the same type e.g. of one of blood, plasma, serum, urine, faeces, sputum, synovial fluid and saliva.

Specifically, the second sample is taken from the subject at a later timepoint, such as within hours or within 1, 2, 3, or 4 days following the first sample.

Specifically, the method described herein may employ an internal standard that is added to the sample optionally before protein precipitation or protein removal.

Specifically, an aliquot of the sample is spiked with an internal standard compound, and the amount of the biomarker is determined by comparing the level of the biomarker to the level of the internal standard compound.

It is preferred that such internal standard is determined with the biomarker side-by-side in the same sample, and can be differentiated from the biomarker resulting in different detection signals. Such internal standard may serve as a positive control to determine whether the assay was suitably carried out. The internal standard may as well serve as a reference to estimate or determine the quantity of the biomarker in the sample e.g., by comparing a detection signal of a predetermined amount of the internal standard to a detection signal of the biomarker in the sample.

Specifically, the internal standard compound is a certain stereoisomer or derivative of the biomarker, or a labelled biomarker, and the method differentiates between the endogenous biomarker comprised in the sample, and such internal standard.

Specifically, the internal standard compound is the biomarker comprising an isotopic label, e.g., suitable for HPLC-MS or HPLC-MS/MS analysis. Preferably, the internal standard compound comprises one or more heavy stable isotopes selected from C¹³, D, N¹⁵, O¹⁷ or O¹⁸, or a combination thereof, in particular substituting one or more of the respective atoms in structure (I). For example, C¹³ may substitute C¹², D may substitute H, N¹⁵ may substitute N¹⁴, and O¹⁷ or O¹⁸ may substitute O¹⁶.

The invention further provides for the use of a diagnostic preparation comprising an immunoagent specifically recognizing a biomarker of structure (I) or a salt thereof, as further described herein, in a method of determining a sepsis condition, wherein the diagnostic preparation further comprises a diagnostic reagent that is a detectable label or a reagent specifically reacting with the immunoagent and/or the reaction product of the immunoagent binding the biomarker. For example, the label may be an enzymatic label, a fluorescent label, or a radioisotope label or tag, indicating an immunoreaction between the immunoagent and the analyte.

Specifically, the diagnostic preparation is provided as a composition or a kit of parts, and optionally further comprises a solid support to immobilize at least one of the immunoagent or the diagnostic reagent.

The invention further provides for the use of an immunoagent specifically recognizing a biomarker for determining a sepsis condition, of structure (I) or a salt thereof, as further described herein and which comprises a detectable label.

Specifically, the biomarker described herein is used as an in vitro marker of a sepsis condition that is conveniently determined by an in vitro method, e.g. in a biological sample of the patient ex vivo.

The invention further provides for an in vitro use of the diagnostic preparation, the immunoagent, or the labelled compound as described herein, in a method described herein.

The invention further provides for a new use of a compound of structure (I) or a salt thereof, as further described herein, as a biomarker of disease.

### BRIEF DESCRIPTION OF THE FIGURES

FIG. 1: Chromatogram showing a BM1 peak as measured in a plasma sample of a healthy human being.

FIG. 2: Chromatogram showing a BM1 peak as measured in a plasma sample of a septic patient

FIG. 3 shows the Area Under the ROC Curve, Example 4.

### DETAILED DESCRIPTION OF THE INVENTION

Unless indicated or defined otherwise, all terms used herein have their usual meaning in the art, which will be clear to the skilled person. Specific terms as used throughout the specification have the following meaning.

Unless indicated otherwise, the term “about” as used herein refers to the same value or a value differing by up to +/−20% of the given value.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The present invention provides for the provision of methods and means for diagnosis, prognosis, prediction, risk assessment and/or risk stratification of a subsequent development of a sepsis condition or a sepsis-related complication in a subject, such as a high-risk patient. Specifically, the biomarker level correlates with the likelihood of a subsequent development of a sepsis condition.

The term “at risk of” as used herein is understood in the following way.

The risk of a sepsis condition is herein understood as being the elevated likelihood of the sepsis condition or occurrence of such sepsis condition in a short timeframe, such as within hours or within 1, 2, 3 or 4 days following the test employing a method described herein, in particular in a subjects who are of a risk population or group including patients who have been diagnosed with an infectious disease but not yet showing symptoms of sepsis, and/or those subjects at higher risk of infection, in particular hospitalized patients and patients with preexisting comorbidities, trauma patients and those with malfunctioning immune systems.

Specifically, the likelihood of the occurrence of a sepsis condition can be assessed on the comparison of the level of the biomarker in the sample with the reference.

The term “onset of disease” is herein understood as the timepoint when a subject develops symptoms of a disease leading to a diagnosis. By predicting the onset of disease, a risk determination is provided which provides for the ability of the trained person to identify a subject that has acquired the disease with high likelihood, such that the subject may receive a respective therapy even before the disease is formally diagnosed. The disease onset is specifically the first appearance of the signs and/or symptoms directly attributable to developing the sepsis condition.

The present invention allows to predict the onset of disease or the disease progression employing a small molecule biomarker. A small molecule biomarker is specifically understood as organic compound of a low molecular weight (<900 daltons) which is a cellular metabolite resulting from a biological process. In particular, the biomarker of structure (I) or a salt of any one of the foregoing.

In specific cases, the condition is sepsis and though the subject might suffer from an infectious disease, the subject does not show at least two early symptoms of sepsis, or is not suffering from sepsis: However, such subject may show only one early symptom of sepsis, and/or may be at risk of developing sepsis. In such case, the biomarker level in a sample of the subject drawn before the subject shows clear symptoms of sepsis, can be indicative of the elevated risk of developing sepsis or septic shock, and an early therapy may be recommended to prevent sepsis development and/or halt sepsis progression.

Early symptoms of sepsis include e.g.:
a fever above (38° C.) or a temperature below 36° C.
resting heart rate higher than approximately 90 beats per minute
breathing rate higher than 20 breaths per minute
pale/mottled skin
altered mentation

Each of such symptoms may indicate a risk of sepsis, in particular, if found in a subject of a high-risk patient group. High risk patients typically have a higher risk of infection, in particular hospitalized patients. Among the high-risk patient groups, also understood as a “risk population”, there are e.g.:
people who underwent biopsy and/or surgery;
people who underwent trauma/accident;
people with pre-existing diseases (e.g. diabetes, cardiovascular insufficiency)
young children (<1 year of age) and seniors (>65 years of age);
people with weaker immune systems, such as those with HIV or those in chemotherapy treatment for cancer;
people being treated in an intensive care unit (ICU) for any cause;
people exposed to invasive devices, such as intravenous catheters or breathing tubes.

Advanced symptoms of sepsis are indicated if one or more of the following signs are found in a subject:
patches of discolored skin
decreased urination
changes in mental ability
low platelet (blood clotting cells) count
problems breathing
abnormal heart functions
chills due to fall in body temperature
unconsciousness
extreme weakness The Quick SOFA Score (quickSOFA or qSOFA) assists health care providers in estimating the risk of morbidity and mortality due to sepsis and is a commonly accepted way to identify patients at high risk for poor outcome with an infection.

Assessment qSOFA Score

| | |
|---|---|
| Low blood pressure (SBP ≤ 100 mmHg) | 1 |
| High respiratory rate (≥22 breaths/min) | 1 |
| Altered mentation (GCS ≤ 14) | 1 |

The score ranges from 0 to 3 points. The presence of 2 or more qSOFA points (and change by 2 points from the pre-existing, non-zero SOFA level) near the onset of infection is associated with a greater risk of death or prolonged intensive care unit stay. These are outcomes that are more common in infected patients who may be septic than those with uncomplicated infection. Based upon these findings, the Third International Consensus Definitions for Sepsis recommends qSOFA as a simple prompt to identify infected patients outside the ICU who are likely to be septic.

According to Singer et al. (The Third International Consensus Definitions for Sepsis and Septic Shock (Sepsis-3); JAMA. 2016; 315(8):801-10), the Sepsis-3 definition is as follows:
Sepsis is defined as life-threatening organ dysfunction caused by a dysregulated host response to infection.
Organ dysfunction can be identified as an acute change in total SOFA score points consequent to the infection.
   The baseline SOFA score can be assumed to be zero in patients not known to have preexisting organ dysfunction.
   ASOFA score ≥2 reflects an overall mortality risk of approximately 10% in a general hospital population with suspected infection. Even patients presenting with modest dysfunction can deteriorate further, emphasizing the seriousness of this condition and the need for prompt and appropriate intervention, if not already being instituted.
In lay terms, sepsis is a life-threatening condition that arises when the body's response to an infection injures its own tissues and organs.
Patients with suspected infection who are likely to have a prolonged ICU stay or to die in the hospital can be promptly identified at the bedside with qSOFA, alteration in mental status, systolic blood pressure 100 mm Hg, or respiratory rate 22/min.
Septic shock is a subset of sepsis in which underlying circulatory and cellular/metabolic abnormalities are profound enough to substantially increase mortality.
Patients with septic shock can be identified with a clinical construct of sepsis with persisting hypotension requiring vasopressors to maintain MAP ≥65 mm Hg and having a serum lactate level >2 mmol/L (18 mg/dL) despite adequate volume resuscitation. With these criteria, hospital mortality is in excess of 40%.

Abbreviations: MAP, mean arterial pressure; qSOFA, quick SOFA; SOFA: Sequential [Sepsis-related] Organ Failure Assessment.

The term “biomarker” as used herein shall refer to a molecule of interest that is a biological marker which is objectively measured and evaluated as an indicator of a normal biological process, pathogenic process, or pharmacological responses to therapeutic interventions. The biomarker referred to herein is a small molecule organic biomarker, and particularly a molecule with the structure depicted in formula (I) or a salt thereof, including those molecules with a specific sterical conformation of formula (I), such as including one or more isomers.

Specifically, the biomarker described herein is of structure (I) shown below:

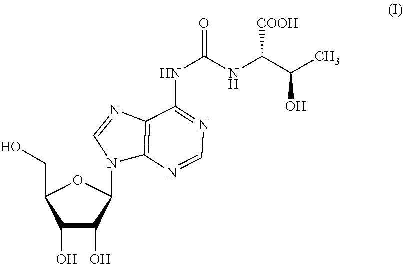

or a salt thereof.

The biomarker, also referred to as BM1, is understood to be of the structure of formula (I), or a salt thereof. For example, BM1 can be determined as an acid or a salt thereof, such as a physiological salt e.g., a sodium salt.

Name: N6-Carbamoylthreonyl-D-adenosine;

Other names: N6-(N-Threonylcarbonyl)adenosine,

N6-Threonylcarbamoyladenosine,

N-{[(9-β-D-Ribofuranosyl-9H-purin-6-yl)amino]carbonyl}-L-threonine.

Molecular formula: C₁₅H₂₀N₆O₈

CAS-number: 24719-82-2.

Deutsch et al. (J. Biol. Chem. 2012, 287:13666-13673); Miyauchi K et al. Nat Chem Biol. 2013 February; 9(2):105-11.

The biomarker may include any respective optical isomer, enantiomer, diastereomer, or racemate, in particular one or more compounds of the structure (I) possibly comprised in the sample as endogenous or naturally-occurring compound(s).

The biomarker described herein may comprise one or more labels, specifically one or more mass tags or mass labels. The term label used in the present context is intended to refer to a moiety suitable to label an analyte for determination. The term label is synonymous with the term tag. Specifically, the term mass label is intended to refer to a moiety that is to be detected by mass spectrometry.

Specifically, the biomarker described herein comprises an isotopic label, which is a non-radioactive stable isotope. Specifically, the isotopic label may be one or more heavy stable isotopes selected from C¹³, D, N¹⁵, O¹⁷ or O¹⁸, or a combination thereof.

Specifically, the biomarker comprising an isotopic label may be used as internal standard compound. A preferred internal standard compound can be added to the sample and analytically differentiated from the target biomarker in the sample.

Specifically, the internal standard compound can be an isotopologue, or can be a compound of structure (I) or a salt thereof, comprising one or more heavy stable isotopes selected from C¹³, D, N¹⁵, O¹⁷ or O¹⁸, or a combination thereof, in particular substituting one or more of the respective atoms in structure (I). For example, C¹³ may substitute C¹², D may substitute H, N¹⁵ may substitute N¹⁴, and O¹⁷ or O¹⁸ may substitute O¹⁶.

An appropriate internal standard can be selected to generate accurate results. Specifically, for MS analysis isotopologues can often be the best choice, as they can very closely match a retention time of the biomarker, and can produce a signal on a different mass channel of a MS detector, which would not interfere with integration of the biomarker signal. Alternatively, a structural isomer of the biomarker structure can be used to produce similar results, or a closely related structure in the same or very similar chemical class can be utilized. Without wishing to be limited by theory, the more structurally similar the internal standard is to the biomarker structure, the more accurate the results will be.

An exemplary internal standard of BM1 comprises the following structure (II):

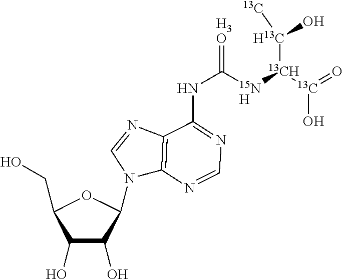

(e.g., available by Toronto Research Chemicals, Canada; Product no: T405562).

Chemical name: N6-(N-Threonylcarbonyl)adenosine-13C4, 15N.

CAS number: 1195030-28-4

Molecular formula: C₁₁¹³C₄H₂₀N₅¹⁵NO₈.

As used herein, “diagnosis” relates to the recognition and (early) detection of a clinical condition of a subject linked to a sepsis condition. Also, the assessment of the severity of the sepsis condition may be encompassed by the term “diagnosis”. “Prognosis” relates to the prediction of an outcome or a specific risk for a subject. This may also include an estimation of the chance of recovery or the chance of a disease progression for said subject.

The methods of the invention may also be used for monitoring, therapy monitoring, therapy guidance and/or therapy control. “Monitoring” relates to keeping track of subject such as an infected or a sepsis patient, and potentially occurring disease progression or complications, e.g. to analyze the progression of the healing process or the influence of a particular treatment or therapy on the health state of the patient.

The term “indicate” as used herein e.g., in the context of indicating an event, such as a disease condition, the risk of a disease condition, disease progression, or treatment success is herein understood as a measure of risk and/or likelihood. Preferably, the “indication” of the presence or absence of an event is intended as a risk assessment, and is typically not to be construed in a limiting way as to point definitively to the absolute presence or absence of said event.

The determination of high or low biomarker levels has turned out to be highly reliable with respect to determining the presence or absence of a risk of sepsis or a sepsis condition when using the reference values described herein, such that the estimation of risk enables an appropriate action by a medical professional.

It was entirely surprising that a level of the biomarker thereof could be correlated with the likelihood of the presence or absence of a subsequent development of a sepsis condition in a high-risk population e.g. in ICU patients. High levels of the biomarker indicate a high severity level and low levels indicate a low severity level. The respective concentrations that determine the reference value, which may be used to assign the respective severity levels, may depend on multiple parameters such as the time point of sample isolation after bacterial infection, the prevalence of an early symptom of sepsis, and the method used for determining the biomarker level in said sample. Accordingly, the method described herein enables a more accurate assessment of the prognosis/risk of a patient depending on the situation of sample isolation and the further information available at that time, for example an increased risk of developing a sepsis condition, sepsis or a specific sepsis-related complication.

The term “immunoagent” as used herein is understood as a molecule that contain an antigen binding site that specifically binds or immunoreacts with an antigen. Preferred immunoagents are antibodies or antigen-binding fragments thereof e.g., monoclonal or polyclonal antibodies or respective antibody fragments. Particularly, antibodies that are specifically binding to the biomarker are employed in an immunoassay for determining the biomarker.

Specific immunoagents may be capture molecules or molecular scaffolds, which are understood as molecules that may be used to bind target molecules or molecules of interest, i.e. analytes such as the biomarker from a sample. Such molecules are shaped adequately, both spatially and in terms of surface features, such as surface charge, hydrophobicity, hydrophilicity, presence or absence of Lewis donors and/or acceptors, to specifically bind the target molecule. Hereby, the binding may, for instance, be mediated by ionic, van-der-Waals, pi-pi, sigma-pi, hydrophobic or hydrogen bond interactions or a combination of two or more of the aforementioned interactions or covalent interactions between the capture molecules or molecular scaffold and the target molecule. Capture molecules or molecular scaffolds may for instance be selected from the group consisting of a nucleic acid molecule, a carbohydrate molecule, a PNA molecule, a protein, a peptide and a glycoprotein, for example, aptamers, DARpins (Designed Ankyrin Repeat Proteins), Affimers and the like.

Specifically, the immunoagent is considered to be specifically recognizing a target antigen such as the biomarker described herein, if its affinity towards the target antigen is at least 100-fold or 1000-fold higher than towards other molecules comprised in the sample containing the biomarker. It is well known in the art how to develop and to select antibodies with a given specificity to recognize a target antigen.

According to a specific aspect, the immunoassay may employ at least one antibody which is labeled, and another antibody that is bound to a solid phase or can be bound selectively to a solid phase. The first antibody and the second antibody can be present dispersed in a liquid reaction mixture, and a first labeling component may be bound to the first antibody, and a second labeling component of said labeling system may be bound to the second antibody so that, after binding of both antibodies to the biomarker to be detected, a measurable signal which permits detection of the resulting sandwich complexes in the measuring solution is generated

According to a specific aspect, the method described herein may be performed as an immunoassay comprising the steps of:

a) contacting the sample with a first antibody or an antigen-binding thereof, specific for a first epitope of the biomarker, and with second antibody or an antigen-binding fragment thereof, specific for a second epitope of the biomarker; and

b) detecting the binding of the first and second antibodies or antigen-binding fragments thereof to the biomarker.

In particular, the first antibody and the second antibody may be present dispersed in a liquid reaction mixture, and wherein a first labelling component is bound to the first antibody, and/or a second labelling component of said labelling system Is bound to the second antibody so that, after binding of both antibodies to at least one biomarker or fragment thereof, a measurable signal which permits detection of the resulting sandwich complexes in the measuring solution is generated.

In specific cases, the method is carried out as sandwich immunoassay, specifically wherein one the antibodies is immobilized on a solid phase, for example, the walls of coated test tubes, microtiter plates, or magnetic particles, and another antibody comprises a detectable label or means enabling for selective attachment to a label, and which serves the detection of the formed sandwich structures.

As used herein, “infection” within the scope of the invention means a pathological process caused by the invasion of normally sterile tissue or fluid by pathogenic or potentially pathogenic agents/pathogens, organisms and/or microorganisms, and specifically relates preferably to infection(s) by bacteria, viruses, fungi, and/or parasites.

The biomarker and/or the immunoreagent described herein may be labelled. As used herein, the term “label” refers to a detectable compound or composition which is conjugated directly or indirectly to another compound, so as to generate a “labelled” compound. The label comprises a detectable moiety. The detectable moiety may be capable of producing, either directly or indirectly, a detectable signal. The label may be detectable by itself (e.g. radioisotope labels or fluorescent labels) or, in the case of an enzymatic label, may catalyze chemical alteration of a substrate compound or composition which is detectable.

The biomarker or immunoreagent described herein can be directly or indirectly conjugated to a label which provides a detectable signal, e.g. radioisotope, fluorescers, enzyme, antibodies, particles such as magnetic particles, chemiluminescers, quantum dots, or specific binding molecules, etc. Preferred labels include, but are not limited to, fluorescent labels, label enzymes and radioisotopes. Suitable labels include, for example, fluorescent or chemiluminescent compounds, such as luciferin, fluorescein isothiocyanate, rhodamine, tetramethylrhodamine, eosin, erythrosin, coumarin, methyl-coumarins, pyrene, Malacite green, stilbene, Lucifer Yellow, CASCADE BLUE®, TEXAS RED®, IAEDANS, EDANS, BODIPY FL, LC Red 640, Cy 5, Cy 5.5, LC Red 705 and OREGON GREEN™.

Suitable labels also include stable isotope labels (e.g. ²H (identical to D), ¹³C, ¹⁵N, etc.), radioactive labels, also called radioisotope labels, (e.g., ¹²⁵I, ³⁵S, ³²P, ¹⁸F, ¹⁴C, ³H, etc.), biological labels, including specific binding molecules (e.g., biotin, streptavidin, digoxin and antidigoxin, etc.), an enzyme, such as alkaline phosphatase, β-galactosidase, or horseradish peroxidase, and other labels.

The term “mass spectrometry” or “MS” as used herein refers to an analytical technique to identify compounds by their mass. There are well-known techniques to quantify the level of a biomarker in the sample by mass.

Generally, MS allows identification of amount and type of compounds (e.g., molecules) present in a sample by measuring the mass-to-charge ratio and abundance of gas-phase ions. MS ionizes chemical compounds to generate charged molecules or molecule fragments and measures their mass-to-charge ratios. The molecular ion and/or molecule fragments are actually charged ions with a certain mass. The mass of the molecular ion and/or fragment divided by the charge is called the “mass-to-charge ratio” (m/z). Since most molecular ions and/or fragments in positive ion mode carry a charge of +1, the mass-to-charge ratio usually represents the molecular weight of the molecular ion and/or fragment. MS produces a mass spectrum for each analyzed compound using a collision cell (called MS/MS mode or MRM mode), wherein the x-axis represents the mass-to-charge ratio and wherein the y-axis represents the signal intensity (abundance) for each of the fragments detected. As will be appreciated by one of skill in the art, and with the help of this disclosure, the mass spectrum produced by a given compound and its product ion spectrum in MS/MS mode or MRM mode is essentially the same every time, and can be regarded as a “fingerprint” of the compound. This fingerprint can be used to identify the compound as well as quantify it reliably and reproducibly.

The y-axis in the mass spectrum can be a relative abundance axis. The peak with the greatest abundance is usually referred to as a “base peak,” and for the purpose of making a relative abundance axis the base peak intensity is set to 100%, such that the entire mass spectrum is normalized to the base peak. In some embodiments, the molecular ion peak can be the base peak. In such embodiments, the entire mass spectrum can be normalized to the molecular ion peak, wherein the molecular ion peak has 100% relative abundance.

According to a specific embodiment of the method described herein, an internal standard is added to a sample from a subject or patient. It will be acknowledged that by said addition of internal standard to the sample, i.e. spiking of the sample, the concentration of the internal standard in the sample is known and, e.g., by determining the area under the peak, i.e. the peak area, of the internal standard in, e.g., an HPLC-mass spectrometric chromatogram the relation between a peak area and a concentration of a substance, e.g. of internal standard and/or the biomarker described herein can thus be calculated, e.g., by calculating the ratio of the peak area of the biomarker described herein and the peak area of the internal standard, which may specifically be the biomarker described herein comprising an isotopic label.

In specific cases of mass spectrometry, the samples can be processed prior to MS analysis, such as immuno-enrichment technologies, methods related to sample preparation and/or chromatographic methods, e.g., the MS can be combined with liquid chromatography (LC), such as high performance liquid chromatography (HPLC) or ultrahigh performance liquid chromatography (UHPLC).

Optional sample preparation methods comprise techniques for lysis, fractionation, digestion of the sample into peptides, depletion, enrichment, dialysis, desalting, alkylation, peptide reduction, protein precipitation and/or extraction (liquid/liquid and solid phase).

The selective detection of the analyte may be conducted with tandem mass spectrometry (MS/MS).

As used herein, the term “subject” or “individual” or “patient” shall refer to a warm-blooded mammalian, particularly a human being.

Specifically, the term “patient” includes mammalian, specifically human, subjects that receive treatment or are at risk of or diagnosed of a specific disease or disorder, particularly those conditions as further described herein. The term “treatment” is meant to include both prophylactic and therapeutic treatment.

Specifically, the subject is a patient who suffers from any disease condition and is at risk of developing sepsis. In particular, the subject may have already been diagnosed as being suffering from an infectious disease, and is optionally receiving antibiotic treatment. The method described herein can therefore be used for determining the onset of a sepsis condition, and/or the monitoring the success of the antibiotic treatment that has been initiated to prevent sepsis disease or disease progression. In such case, the biomarker level can be used as indicator of the likelihood of success of an antibiotic treatment. It can also be decided, whether antibiotic treatment should be continued, because it is likely that it is working or improving the health state of the patient, or whether it should be changed.

The term “reference level” as used herein is understood in the following way.

The term “level” is herein understood as the absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker. Specific levels are provided as a value of the amount representing the concentration, and specifically expressed as weight/volume; w/v, or expressed as fold change of the biomarker in the sample.

Specifically, the reference level is a threshold, also understood as a cut-off value indicating a biomarker concentration for the respective risk or the severity of disease. The respective concentrations that determine the threshold values depend on multiple parameters such as the time point of sample isolation and the assay or detection used for determining the biomarker level in said sample.

An elevated or “higher” level is e.g., significantly higher than the reference. The term “significant” as used herein shall refer to at least a two-fold higher amount of the standard deviation, preferably at least a three-fold difference. With respect to a specific reference value, such as derived from a standard, training data or threshold, a significant elevated or increased amount is particularly understood to refer to an at least 1.5-fold higher amount, preferably at least 2- or 3-fold difference.

As used herein, the term “cutoff value” refers to a threshold value which distinguishes subjects who are suffering from a disease or disease condition from patients and/or subjects who are not suffering from the disease or disease condition, and in particular distinguishes subjects who are at a certain (e.g. high risk) of disease or disease progression from patients and/or subjects who are not at such risk of disease or disease progression e.g., healthy subjects or subjects that are not infected by a pathogen.

As used herein, the terms “reference” or “control” may be an agent or value that allows comparison to the result of such diagnostic assays so that conclusions may reasonably be drawn based on differences or similarities observed. Suitable values are e.g. predetermined levels of predictability for disease or disease progression, such as obtained from biological samples of one or more patients that later developed severe symptoms of disease or disease progression. In this regard, positive predictive values or negative predictive values may be used as a reference.

The relevant reference levels can be determined by well-known methods e.g. based on extensive data which can be routinely obtained by comparing samples from diseased patients with healthy subjects or subjects not suffering from such disease. Such references are understood as population averages levels, for example mean biomarker population values, whereby patients that are diagnosed as sepsis patient may be compared to a control population, wherein the control group preferably comprises more than 10, 20, 30, 40, 50 or more subjects. The Non-Diseased concentration levels in plasma/serum/blood of different populations (Caucasians, Asians, Africans etc. . . . ) might vary from one kind to another.

Appropriate normal reference levels of the biomarker may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a sepsis condition state, phenotype, or lack thereof in a certain age or gender group).

Typically, the reference value to predict a disease condition in a sample of a patient not suffering from said disease condition is lower than the reference value to predict disease progression. The latter can be e.g., at least 2-fold, 3-fold, 4-fold, 5-fold, 10-fold, 15-fold, or even at least 20-fold increased.

Functional assays can be used to indicate statistically significant values for use as reference levels or cut-offs according to established techniques. Laboratories are capable of independently establishing an assay's functional sensitivity by a clinically relevant protocol.

The term “sample”, or “biological sample” or “specimen”, means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, blood, blood plasma, serum, skin, epidermal tissue, adipose tissue, aortic tissue, liver tissue, urine, sebum, cell samples, sputum, synovial fluid or saliva.

The present biomarker can be used as a predictive marker, e.g. as a stand-alone diagnostic or in combination with further diagnostic measures, such as the determination of further markers (e.g. in a diagnostic combination kit).

According to certain embodiments, the method described herein additionally comprises determining a level of at least one additional biomarker in a sample from said patient, wherein the at least one additional biomarker preferably is procalcitonin (PCT) or fragment(s) thereof and/or lactate.

Specific methods to detect the biomarker in a sample may be selected from the group consisting of HPLC, UHPLC or UPLC combined with mass spectrometry (MS) (in particular combined with UV detection or fluorescence detection), luminescence immunoassay (LIA), radioimmunoassay (RIA), chemiluminescence- and fluorescence-immunoassays, enzyme immunoassay (EIA), Enzyme-linked immunoassays (ELISA), luminescence-based bead arrays, magnetic beads based arrays, protein microarray assays, rapid test formats e.g., immunochromatographic strip tests, and automated systems/analyzers.

In particular, a sandwich format can be used. For example, one or more binder (e.g., among them an immunoagent specifically recognizing the biomarker) is conjugated to a substrate prior to the contacting with a biological sample. The one or more binder may be conjugated to a detectable label to serve as a detection molecule. In other embodiments, the one or more binder is conjugated to a detectable label. In this configuration, the one or more binders may be conjugated to a substrate prior to the contacting with the biological sample to serve as a capture agent. Furthermore, the one or more binder can be conjugated to a substrate prior to the contacting with the biological sample, and/or the one or more binder is conjugated to a detectable label. In such cases, the one or more binder can act as either or both of a capture agent and a detection agent.

According to a specific aspect, a software system can be employed, in which a machine learning algorithm is evident, preferably to identify hospitalized patients at risk for a sepsis condition or septic shock using data from electronic health records (EHRs). A machine learning approach can be trained on a random forest classifier using EHR data (such as labs, biomarker expression, vitals, and demographics) from patients. Machine learning is a type of artificial intelligence that provides computers with the ability to learn complex patterns in data without being explicitly programmed, unlike simpler rule-based systems.

A diagnostic preparation as further described herein may comprise an immunoagent specifically recognizing the biomarker as described herein and a further diagnostic reagent, which is a detectable label or a reagent specifically reacting with the immunoagent and/or the reaction product of the immunoagent binding the biomarker.

The diagnostic preparation optionally comprises the immunoagent specifically recognizing the biomarker of the invention and the further diagnostic reagent in a composition or a kit of parts.

The term “diagnostic kit” as used herein refers to a kit or set of parts, which in combination or mixture can be used to carry out the measurement/detection of one or more analytes or markers to determine a disease or disease condition, or to predict the disease and particularly the onset of disease, or the disease progression. In particular, the kit contains at least a detection molecule and/or a binder (e.g. an immunoagent), wherein the detection molecule and/or the binder specifically recognizes the marker or the respective analyte, or a reaction product of such marker or analyte. In addition, various reagents or tools may be included in the kit. The diagnostic kit preferably comprises all essential components to determine the amount of the biomarker in the biological sample, optionally without common or unspecific substances or components, such as water, buffer or excipients that may be conveniently added when performing the analysis. The diagnostic kit may comprise any useful reagents for carrying out the subject methods, including substrates such as microbeads or planar arrays or wells, reagents for biomarker isolation, detection molecules directed to specific targets, detectable labels, solvents, buffers, linkers, various assay components, blockers, and the like.

A kit may also include instructions for use in a diagnostic method. Such instructions can be, for example, provided on a device included in the kit, e.g. tools or a device to prepare a biological sample for diagnostic purposes, such as separating a cell and/or protein containing fraction before determining a marker. The kit may conveniently be provided in the storage stable form, such as a commercial kit with a shelf-life of at least 6 months. The storage stable kit can be stored preferably at least 6 months, more preferably at least 1 or 2 years. It may be composed of dry (e.g. lyophilized) components, and/or include preservatives.

The preferred diagnostic kit is provided as a packaged or prepackaged unit, e.g. wherein the components are contained in only one package, which facilitates routine experiments. Such package may include the reagents necessary for one or more tests, e.g. suitable to perform the tests of a series of biological samples. The kit may further suitably contain a biomarker preparation as a standard or reference control, which may be the biomarker comprising a label.

The diagnostic composition may be a reagent ready-to-use in a reaction mixture with the biological sample, or a conserved form of such reagent, e.g. a storage-stable form such as lyophilized; snap-frozen (e.g. in liquid nitrogen), ultra-low temperature storage (−70° C. and −80° C.), cold-storage (−20° C. and 5° C.) and controlled room temperature (15° C.-27° C.); standard sample storage as e.g. glycerol-stocks, tissue paraffin-blocks, (buccal) swabs and other standard biological sample storage methods, which conserved form of a reagent can be reconstituted or prepared to obtain a ready-to-use reagent. Such ready-to-use reagent is typically in the form of an aqueous solution, specifically (physiological) buffer conditions (e.g. EDTA buffered, phosphate buffer, HBSS, citrate buffer etc.).

The further diagnostic reagent comprised in the diagnostic preparation (in particular the composition or diagnostic kit) can be a reagent specifically reacting with the immunoagent and/or the reaction product of the immunoagent binding the biomarker. An appropriate diagnostic reagent is suitably used for performing an immunoassay for diagnosing or monitoring, in a subject, a sepsis condition. The appropriate diagnostic reagent can be a solvent, a buffer, a dye, an anticoagulant, a ligand that specifically binds to the immunoagent and/or the reaction product of the immunoagent binding the biomarker described herein.

Specifically, the invention provides for a diagnostic preparation of an immunoagent binding the biomarker described herein, optionally containing the immunoagent with a label and/or a further diagnostic reagent with a label, such as a reagent specifically recognizing the immunoagent or an immune complex of the immunoagent and the biomarker, and/or a solid phase to immobilize at least one of the immunoagent and the diagnostic reagent.

The immunoagent or the diagnostic reagent can be directly labeled or indirectly labeled. The indirect label may comprise a labeled binding agent that forms a complex with the immunoagent or diagnostic reagent.

Preferred diagnostic preparations or assays comprise the immunoagent specifically recognizing the biomarker described herein immobilized on a solid phase, e.g. latex beads, gold particles, etc., e.g. to test the presence and amount of the biomarker in a sample to be tested.

The present invention is further illustrated by the following examples without being limited thereto.

### EXAMPLES

### Example 1: Materials & Methods for the Detection of BM1 in Serum, Plasma and Urine

The biomarker of structure (I) as described herein is herein also referred to as BM1. According to this Example, BM1 was detected by HPLC-MS/MS on a reversed phase (RP)-column after protein precipitation with e.g. acetonitrile or methanol from serum plasma or blood or by dilution of urine.

Equipment:

To detect BM1 in a sample of a subject, the following equipment is used.

TABLE 1
Equipment used in the Examples

| | |
|---|---|
| Instrument/material | Type/Producer |

Workstation “XEVO1”

| | | |
|---|---|---|
| UPLC Pump | ACQUITY I Class Plus | Waters |
| | Binary Solvent Manager | Corporation, USA |
| Autosampler/Sample | ACQUITY I Class Plus | Waters |
| Manager | Sample Manager FTN-I | Corporation, USA |
| Sample Organizer | ACQUITY Sample Organizer | Waters |
| | | Corporation, USA |
| Column Oven/Column | ACQUITY Column Manager | Waters |
| Manager | | Corporation, USA |
| Mass Spectrometer | XEVO TQ-XS | Waters |
| | | Corporation, USA |

Sample Preparation:

To determine the concentration of BM1 in healthy human subjects, serum, plasma and urine samples are derived from human or non-human animal subjects. The samples are prepared as follows.

a) Sample Preparation Procedure

TABLE 2
Sample preparation

| Step | Sample preparation procedure (step by step) |
|---|---|
| 1 | Transfer of 50 μL of each plasma/serum/blood sample into |
| | a conical glass sample vial (volume approx. 5 mL) |
| 1a | For URINE Samples ONLY: Addition of 950 μL of water |
| 1b | Vortexing with a DVX-2500 Multi-tube vortexer (2500 rpm) |
| | for about 1 minute |
| 1c | Transfer of 50 μL of diluted samples from STEP 1b into a |
| | conical glass sample vial (volume approx. 5 mL) |
| 2 | Addition of 20 μL of internal standard working solution |
| | (continued for all matrices plasma/serum/blood/urine) |
| 3 | Addition of 100 μL ACN for protein precipitation |
| 4 | Vortexing with a DVX-2500 Multi-tube vortexer (2500 rpm) |
| | for about 1 minute |
| 5 | Centrifugation with e.g. Megafuge 16 at 4000 rpm for 2 minutes |
| 6 | Add 100 μL of water to labelled and empty (conical) |
| | auto-sampler vials |
| 7 | Transfer of about 50 μL into auto-sampler vials without |
| | wetting of the pipette tip |
| 8 | Crimping the vials with appropriate vial caps |
| 9 | Vortexing with a DVX-2500 Multi-tube vortexer (2500 rpm) |
| | for about 1 minute |
| 10 | [Analysis within max. 85 hours]; Store under auto-sampler |
| | conditions at about 25° C. until analysis |

b) Chromatography

The samples prepared for analysis as described in a) are subsequently subjected to HPLC chromatography according to the following parameters:

TABLE 3
Chromatographic and Auto-Sampler Parameters

| Parameter | Scheduled range/description |
|---|---|
| Mobile phase solvent A | 0.1% Formic Acid in Water |
| Mobile phase solvent B | 0.1% Formic Acid in 90% Acetonitrile |
| Chromatographic run | 0.0-0.5 min isocratic: 0% B |
| | 0.5-3.0 min linear gradient: 0% B → 25% B |
| | 3.01-4.0 min isocratic: 100% B |
| | 4.01-5.2 min isocratic: 0% B |
| Flow | 1 mL/min |
| Injection volume | 3 μL |
| Column | YMC Pack Pro C8, 100 × 3 mm, 12 nm |
| | (YMC, Japan) |
| Column temperature | 60° C. |
| Typical Column | about 350 bar after injection on XEVO1, |
| Pressure | method-specific, informative only |
| Retention time BM1 | 2.6 min ± 20% |
| and Internal Standard | |

Detection of BM1 Using Mass Spectrometry

The prepared samples are subsequently subjected to UPLC-mass spectrometry, according to the parameters described in Table 4.

TABLE 4
MS-Detection (XEVO TQ-XS using UNISpray as ion source)

| Parameter | Scheduled range/description |
|---|---|
| Parameters as defined | Scheduled range/description |
| in MS method .exp | |
| MS Ionisation mode | US+ (UNISpray in positive ion mode) |
| and polarity | |
| MS detection mode | MRM (Multiple reaction monitoring) |
| Auto dwell | On |
| Dwell time [s] | About 0.053 (per peak) |
| Retention window [min] | 1-3 (defines acquisition time for |
| | this function compared to sample |
| | manager injection time) |
| Cone voltage | 20 V |
| Parameters as defined | Scheduled range/description |
| in MS Tune method .ipr | |
| Impactor voltage | 3.0 kV |
| Desolvation temperature | 600° C. |
| Desolvation gas flow | 1200 [L/h] |
| Cone gas flow | 200 [L/h] |
| Nebuliser | pressure = 7 bar (fixed parameter) |
| Collision gas flow | 0.2 [mL/min] |
| Vertical position | approx. 5.5 units |
| Impactor position | 4¼ units, needle tip about 1 mm visible |
| Quadrupole resolution | 3.0/15.0/0.5 AND |
| defined as: | 2.7/15.0/0.5 |
| Analyser LM Resolution 1/ | |
| Analyser HM Resolution 1/ | |
| Ion energy 1 | |
| Analyser LM Resolution 2/ | |
| Analyser HM Resolution 2/ | |
| Ion energy 2 | |

Evaluation and Calculation of Results

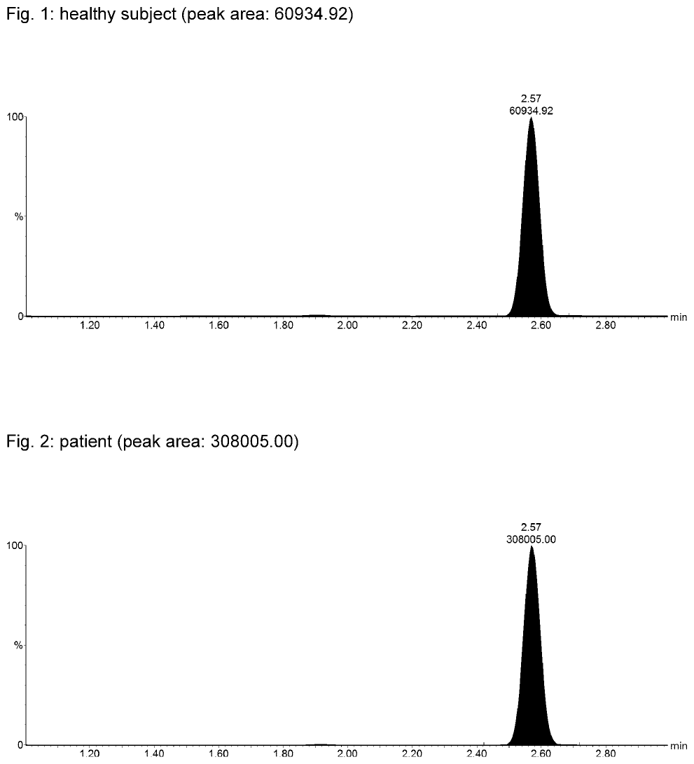

An exemplary chromatogram showing the BM1 content in a plasma sample of a healthy human being is provided as FIG. 1. An exemplary chromatogram showing the BM1 content in a plasma sample of a human being suffering from sepsis is provided as FIG. 2.

Rounding Procedure

Concentration data fed into and retrieved from the chromatographic data system (CDS) are rounded to five significant digits. Further calculations in the spreadsheet are performed and subsequently reported with rounded three significant digits. Accuracy and coefficients of variation (CV) are reported with one digit.

Note Referring to the Rounding Procedure:

The last digit reported is up-rounded if the subsequent digit is equal or greater than “5”.

Regression and Statistics

Based on calibration standards the calibration curve fitting is established using the data processing software by means of peak area ratios (analyte BM1/internal standard). Analyte BM1 concentrations are evaluated using an internal standard method.

For HPLC-MS/MS analysis an adequately and stable regression model is used. Therefore, the quadratic regression model with weighting factor 1/conc is set as default. It is used in order to avoid changes in regression model that could occur, especially if a method is used over months or years in different projects.

The concentrations of the analyte BM1 are determined using following regression model, weighting factor and formula:

| | Regression | Weighting | |
|---|---|---|---|
| Analyte | model | factor | Formula for concentration |
| BM1 | y = ax² + bx + c | 1/conc. | $\begin{matrix}concentration ( x ) = \\ \frac{\begin{matrix}- b \pm \\ \sqrt{{b}^{2} - 4 a ( c - peak area ratio ( y ) )}\end{matrix}}{2 a}\end{matrix}$ |

Based thereon mean values, precision results (in terms of CVs) and accuracies (formula shown below) are calculated using a spreadsheet.

$$accuracy ( \% ) = \frac{calculated concentration}{expected coocentration} * 100$$

$$\begin{matrix}standard deviation = \\ \sqrt{\frac{1}{N - 1} {\sum }_{i = 1}^{N} {( {x}_{i} - \overline{x} )}^{2}}\end{matrix} \qquad \begin{matrix}{x}_{i} & calculated concentration \\ \overline{x} & mean calculated concentration \\ N & number of values \\ i & index of values\end{matrix}$$

$$relative standard deviation ( \% ) = \frac{s t a ndard deviation}{mean calculated concentration} ⋆ 100$$

Appropriate publications about statistical models are introduced in e.g.:
Green, J. R., Statistical Treatment of Experimental Data (Elsevier, New York, 1977), page 210 ff
Lothar Sachs, Angewandte Statistik—Anwendung statistischer Methoden (Springer, Berlin, Heidelberg, New York, Tokyo 1984)

Software

| | |
|---|---|
| Data acquisition | MassLynx 4.2 SCN 982 or higher (Waters |
| | Corporation, USA) |
| Data processing | TargetLynx 4.2 or higher (Waters Corporation, USA) |
| Statistics and | 1. TargetLynx 4.2 or higher (Waters Corporation, |
| calculations | USA) |
| | 2. Excel (Microsoft Corporation, USA) |

Detection of PCT

To compare with PCT levels, PCT was determined by commercial assays, for example Elecsys BRAHMS PCT, Procalcitonin (Roche Diagnostics GmbH, Mannheim, Germany).

### Example 2: Determination of BM1 Levels in Healthy Human or Non-Human Animal Subjects

BM1 Levels in Healthy Human Subjects.

The samples were prepared as described above and the level of BM1 in said samples was determined according to the method described in Example 1.

In healthy human subjects, the concentration of BM1 in urine is about 20 to 100 times higher than the concentration of BM1 in plasma concentration. Therefore, the reference level in urine is about 20-100 higher than in serum or plasma.

The reference levels based on data obtained from a healthy or non-septic reference (negative control—heart failure) population are as follows:

Reference level in urine is at about 2500-4500 ng BM1/mg Creatinine.

Reference level in plasma or serum is at about 15-40 ng BM1/mL.

BM1 Levels in Healthy Non-Human Animal Subjects.

The concentration of BM1 in healthy non-human animal plasma or serum was tested and compared to healthy human levels. Two to three different individuals or pools of rat, dog, rabbit, hamster, monkey and mouse was tested. Rat, dog, rabbit, hamster, monkey and mouse had very similar levels compared to humans.

Some samples of calves, lama, alpaca, goat, and beef showed concentration levels of about 10-30 ng/mL, some septic calves showed concentration levels of about 40-80 ng/mL for BM1.

### Example 3: Comparison of BM1 Levels in Healthy Subjects with Subjects at Risk of Suffering from Sepsis

392 plasma/serum samples were derived from 129 human patients suffering from sepsis or septic shock, or from non-septic patients diagnosed with heart failure (=control group).

The samples were prepared as described above and the level of BM1 in said samples was determined according to the method described in Example 1.

Number of patients, sepsis vs. cardiogenic disease (control)

| Disease | N |
|---|---|
| Cardio | 133 |
| Sepsis | 259 |
| total | 392 |

Results

The level of BM1 in the plasma or serum of patients suffering from sepsis, or septic shock was significantly higher than in the respective samples of patients not suffering from a sepsis condition.

### Example 4: Comparing Performance of BM1 to PCT

BM1 and PCT levels were determined as described in Example 1.

Receiver Operating Characteristics

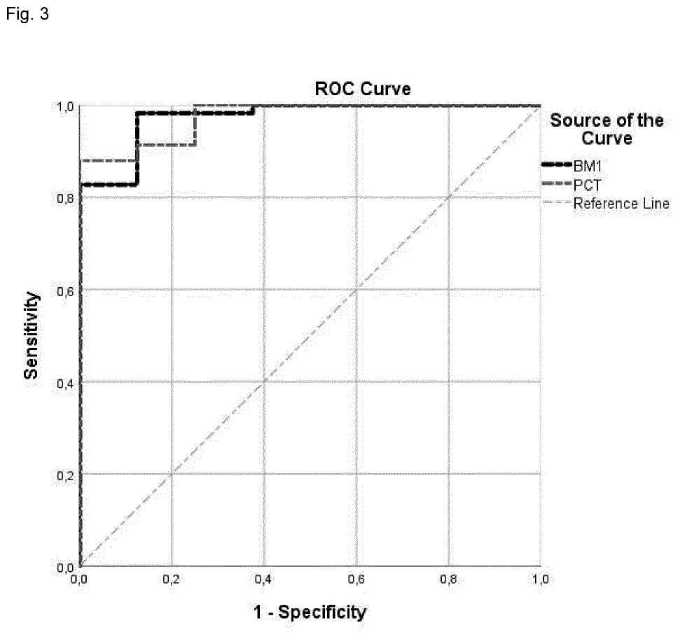

Area Under the ROC Curve (FIG. 3)

| | | | | Asymptotic 95% | |
|---|---|---|---|---|---|
| | | | | Confidence Interval | |
| Test Result | | Std. | Asymptotic | Lower | Upper |
| Variable(s) | Area | Error^{a} | Sig.^{b} | Bound | Bound |
| BM1 | 0.974 | 0.023 | 0.000 | 0.929 | 1.019 |
| PCT | 0.974 | 0.020 | 0.000 | 0.935 | 1.013 |

^{a}Under the nonparametric assumption
^{b}Null hypothesis: true area = 0.5

Matrix: Sensitivity/Specificity—Biomarker 1

Cutoff value used: 32 ng/mL (calculated)

| BM1 | | N | | | N | Sum |
|---|---|---|---|---|---|---|
| True positives | TP | 57 | False positives | FP | 1 | 58 |
| False negatives | FN | 1 | True negatives | TN | 7 | 8 |

| | | | | |
|---|---|---|---|---|
| Positives | 58 | Negatives | 8 | 66 |

Matrix: Sensitivity/Specificity—Procalcitonin

Cutoff value used: 0.5 ng/mL (according to literature)

| PCT | | N | | | N | Sum |
|---|---|---|---|---|---|---|
| True positives | TP | 54 | False positives | FP | 2 | 56 |
| False negatives | FN | 4 | True negatives | TN | 6 | 10 |

| | | | | |
|---|---|---|---|---|
| Positives | 58 | Negatives | 8 | 66 |

Summary of Indices

| | short | Biomarker 1 | | Procalcitonin | |
|---|---|---|---|---|---|
| Sensitivity, true | TPR | 98.28 | % | 93.10 | % |
| positive rate | | | | | |
| Specificity, true | TNR | 87.50 | % | 75.00 | % |
| negative rate | | | | | |
| Positive predictive | PPV | 98.28 | % | 96.43 | % |
| value, precision | | | | | |
| Negative predictive value | NPV | 87.50 | % | 60.00 | % |
| False positive rate | FPR | 12.50 | % | 25.00 | % |
| False negative rate | FNR | 1.72 | % | 6.90 | % |
| False discovery rate | FDR | 1.72 | % | 3.57 | % |
| Accuracy | ACC | 96.97 | % | 90.91 | % |
| F1 score | F1 | 98.28 | % | 94.74 | % |
| Matthews correlation | MCC | 0.8599 | | 0.6356 | |
| coefficient | | | | | |
| Informedness (Youden's | YI | 0.8578 | | 0.6810 | |
| index) | | | | | |
| Markedness (Δp) | Δp | 0.8578 | | 0.5643 | |

### Example 5: Bioanalytical Characterization of BM1 as Biomarker

Comparison of Plasma Versus Urine (Ng BM1/Mg Creatinine)

The following isotopically labelled internal standard is used to determine the quantity of BM1 (1000 ng/mL): N6-(N-Threonylcarbonyl)adenosine-13C4,15N (Compound of structure/formula (II)).

Plasma (or serum) and urine samples from septic patients and control patients are analysed and quantified.

BM1 was found to be very hydrophilic and the urine concentration in healthy volunteers is about 20 to 100 times higher than in plasma/serum. The urine concentration in septic patients can be very high. Reference levels of BM1 in urine of septic patients are at about 5000-16000 ng/mg Creatinine (healthy see example 2).

BM1 Levels in Human Newborns with and without Sepsis

Plasma samples of newborns (day 1-10) without and with sepsis were analysed. BM1 has proven to be a reliable marker of disease. PCT in newborns is practically not used by clinicians for this patient group because PCT levels are very high right after birth in plasma or serum (about 10-30 ng/mL) and are reduced within 4-6 days to a normal concentration (cut off for sepsis for PCT at 0.5 ng/mL in plasma/serum); hence the PCT level is not stable within the first 4-6 days after birth and therefore not characteristic.

BM1 was determined in newborn samples (with and without sepsis); the concentration levels for healthy were at around 80-120 ng/mL (n=6) and in septic newborns elevated levels up to 300 ng/mL (n=7).

Further Studies:

BM1 determination in following specific patient groups comparing with PCT results of the same patients:

Patients with multiple trauma

Patients suffering from severe burns

Patients after major surgery

Malaria patients

Specific tumor patients

### Example 6: ELISA for the Detection of BM1

An immunological test is provided which is fitting to clinical lab equipment for fast analysis. This exemplary ELISA can be used to determine BM1 in plasma or urine using a BM1 specific immunoagent. A monoclonal antibody specifically recognizing BM1 can be provided by commercial sources, such as by custom design. Alternatively, polyclonal non-human animal (e.g. rabbit or sheep) antibodies can be used.

### Example 7: Internal Standard for HPLC-MS/MS

An isotopically labelled BM1 is used as internal standard for quantitative bioanalysis of the above mentioned analysed human plasma and urine samples (example 1):

¹³C₄, ¹⁵N₁-BM1 (5 mass units higher molecular weight).

The structure is of formula (II) as further described herein.

1. A method of predicting a sepsis condition in a subject, comprising:
a. determining a level of a biomarker in a sample of said subject, wherein the biomarker is of structure (I): 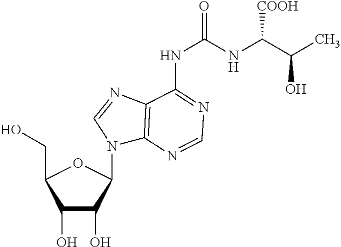
or a salt thereof;
and
b. comparing said level to a predetermined reference value of the biomarker, wherein an elevated biomarker level is indicative of the risk of the sepsis condition.

2. The method of claim 1, wherein the reference level is the level of the biomarker in a subject or a group of subjects not being at said risk, or a threshold level indicating the sepsis condition.

3. The method of claim 1, wherein the sepsis condition is any one or more of sepsis diseases selected from the group consisting of systemic inflammatory response syndrome (SIRS), sepsis, septic shock and multiple organ dysfunction syndrome (MODS).

4. The method of claim 1, wherein the biomarker is determined applying an analytical method selected from the group consisting of
(i) an immunoassay, such as enzymatic immunoassay, lateral flow immunochromatographi c assay, fluorescent immunoassay,
radioimmunoassay, and magnetic immunoassay;
(ii) chromatography, such as HPLC chromatography, UPLC chromatography, gas chromatography (GC) or thin layer chromatography,
(iii) capillary electrophoresis, and
(iv) mass spectrometric analysis, such as SELDI, MALDI, MALDI-Q TOF, MS/MS, TOF-TOF and ESI-Q-TOF, or NMR spectroscopy.

5. A method of monitoring a sepsis disease in a subject, comprising:
a. determining the level of a biomarker in a sample of a subject at a first time point, wherein the biomarker is as defined in claim 1; and
b. determining the level of the biomarker in a sample of the same subject at a later second time point,
wherein an increase in the level of the biomarker between the first and second time points is indicative of the sepsis disease progression.

6. A method of monitoring the effectiveness of at least one treatment applied to a subject for a sepsis condition, comprising:
a. determining the level of a biomarker in a sample of a subject at a first time point, wherein the biomarker is as defined in claim 1; and
b. determining the level of the biomarker in a sample of the same subject at a later second time point, and
wherein a decrease in the level of the biomarker between the first and second time points is indicative of treatment success.

7. The method of claim 1, wherein the sample is body fluid selected from the group consisting of blood, plasma, serum, urine, faeces, sputum, synovial fluid and saliva.

8. The method of claim 1, wherein the sample is spiked with an internal standard compound, and the amount of the biomarker is determined by comparing the level of the biomarker to the level of the internal standard compound, preferably wherein the internal standard compound is the biomarker comprising an isotopic label.

9. The method of claim 8, wherein the internal standard is of structure (I) or a salt thereof, which comprises a detectable label, preferably wherein at least one of the atoms C, H, N, or O, is substituted for the respective heavy isotope C¹³, D, N¹⁵, O¹⁷ and O¹⁸.

10. Use of a diagnostic preparation in a method of determining a sepsis condition, wherein the diagnostic preparation is provided as a composition or a kit of parts, and comprises the following components:
a. an immunoagent specifically recognizing a biomarker as defined in claim 1;
b. a further diagnostic reagent being a detectable label or a reagent specifically reacting with the immunoagent and/or the reaction product of the immunoagent binding the biomarker; and
c. optionally further comprising a solid support to immobilize at least one of the immunoagent or the diagnostic reagent.

11. Use of an immunoagent specifically recognizing a biomarker as defined in claim 1 in a method of determining a sepsis condition, wherein the immunoagent comprises a detectable label.

12. Use of a compound of structure (I): 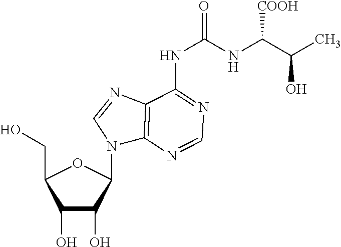
or a salt thereof, as a biomarker of a sepsis condition.

13. A method of predicting a sepsis condition in a subject not suffering from said sepsis condition, comprising:
a. determining a level of a small molecule biomarker in a sample of said subject, and
b. comparing said level to a predetermined reference value of the biomarker,
wherein an elevated biomarker level is indicative of the risk of the sepsis condition onset, and wherein the biomarker is as defined in claim 1.

14. A method of monitoring a sepsis condition in a subject not suffering from said sepsis condition, comprising:
a. determining the level of a small molecule biomarker in a sample of a subject at a first time point, and
b. determining the level of the biomarker in a sample of the same subject at a later second time point,
wherein an increase in the level of the biomarker between the first and second time points is indicative of a sepsis condition onset, and wherein the biomarker is as defined in claim 1.
